(19) United States
(12) Patent Application Publication
(10) Pub. No.: US 2007/0160560 A1
(43) Pub. Date: Jul. 12, 2007

(54) Cosmetic composition comprising at least one fixative polymer and at least one surfactant chosen from ionic and nonionic surfactants

(76) Inventors: Ludivine Laurent, Courbevoie (FR); Marie-Claude Combarieu, Soisy Sous Montmorency (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/589,073

(22) Filed: Oct. 30, 2006

Related U.S. Application Data
(60) Provisional application No. 60/761,389, filed on Jan. 24, 2006.

(30) Foreign Application Priority Data
Oct. 28, 2005 (FR) 05 11093

(51) Int. Cl. A61K 8/81

(52) U.S. Cl. 424/70.15; 424/70.16

(57) ABSTRACT

Disclosed herein is a cosmetic composition for treating keratin fibers, for example human keratin fibres such as the hair, comprising, in a cosmetically acceptable medium, at least one anionic fixative polymer comprising as monomers at least a vinyl lactam, an ethylenically unsaturated carboxylic acid, and an acrylate chosen from alkyl acrylates and alkyl methacrylates whose alkyl moiety contains at least 6 carbon atoms, and at least one surfactant chosen from ionic and nonionic surfactants;
wherein the composition has a viscosity greater than 2 poise (200 cps) at 25° C. at a shear rate of 1 s⁻¹.

This application claims benefit of U.S. Provisional Application No. 60/761,389, filed Jan. 24, 2006, the contents of which are incorporated herein by reference. This application also claims benefit of priority under 35 U.S.C. § 119 to French Patent Application No. FR 05 11093, filed Oct. 28, 2005, the contents of which are also incorporated herein by reference.

Disclosed herein is a cosmetic composition for treating keratin fibers, for example, human keratin fibers such as the hair, comprising at least one fixative polymer and at least one surfactant chosen from ionic and nonionic surfactants. Further disclosed herein is the use of this composition for shaping and/or retaining the hairstyle, and a cosmetic treatment method employing this composition.

In the field of hairstyling, such as among hair products intended for the shaping and/or retention of the hairstyle, hair compositions may be composed of a solution, such as an alcoholic or aqueous solution, and at least one fixative polymer, in a blend with various cosmetic adjuvants.

These compositions may take the form of hair gels, hair lotions, or hair mousses, which may be applied to wet hair prior to blow-drying or other drying operation.

Hair gels may comprise at least one thickening polymer or gelling agent in combination with at least one fixative polymer, which may function to form a film on the surface of the keratin fibers to be fixed, in order to produce bonding points between these fibers.

However, the application of these hair gels may lead, more or less rapidly, to the formation of unattractive residues on the hair, which are a result of the fragility or friability over time of the film responsible for fixing, with partial deaggregation of the bonding points.

Moreover, hair gels may be difficult to distribute in the form of sprays in aerosol devices or in pump dispenser bottles.

There is therefore a need to find cosmetic compositions, such as for hair styling, which may make it possible to minimalize the formation of unattractive residues while imparting to the body of hair a high level of fixing, in order to obtain satisfactory shaping and/or retention of the hairstyle.

The present inventors have now discovered that, by combining at least one fixative polymer and at least one surfactant chosen from ionic and nonionic surfactants, it may be possible to minimize the formation of residues on the hair and/or to obtain satisfactory cosmetic properties.

The combination disclosed herein also may make it possible to provide styling gels with a prehensile facility and with a satisfactory gel texture without the need for addition of a thickening polymer or a gelling agent.

Moreover, the gels obtained may be more readily sprayable, i.e., may be easier to distribute in spray form.

Thus, disclosed herein is a cosmetic composition for treating fibers, for example human keratin fibres such as the hair, comprising the combination disclosed above and discussed in detail below.

Also disclosed herein is the use of the composition for shaping and/or retaining the hairstyle.

Another embodiment disclosed herein is an aerosol device comprising the cosmetic composition disclosed herein.

Also disclosed herein is a cosmetic treatment method employing the composition disclosed herein.

Additional subject matter, features, aspects and advantages of the present disclosure will emerge more clearly from a reading of the description and examples which follow.

Disclosed herein is a cosmetic composition for treating keratin fibers, for example human keratin fibers such as the hair, comprising, in a cosmetically acceptable medium.

at least one anionic fixative polymer comprising as monomers at least a vinyl lactam, an ethylenically unsaturated carboxylic acid, and an acrylate chosen from alkyl acrylates and alkyl methacrylates, whose alkyl moiety comprises at least 6 carbon atoms, and

at least one surfactant chosen from ionic and nonionic surfactants;

wherein the composition exhibits a viscosity greater than 2 poise (200 cps) at 25° C. at a shear rate of 1 s⁻¹.

The viscosity of the cosmetic composition may be determined using an RS600 rheometer from Thermoelectron.

The viscosity of the cosmetic composition may be such that there is no need to add a thickening polymer or a gelling agent.

The at least one ionic surfactant used in the cosmetic composition may be chosen from cationic surfactants.

Examples of cationic surfactants which can be used in the cosmetic composition disclosed herein include, for example, the salts of optionally polyoxyalkylenated primary, secondary, and tertiary fatty amines, quaternary ammonium salts, and mixtures thereof.

Examples of quaternary ammonium salts include, for example:

those having formula (I) below: 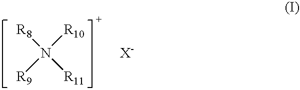
in which

the radicals R₈ to R₁₁, which may be identical or different, are chosen from linear or branched aliphatic radicals containing 1 to 30 carbon atoms and aromatic radicals, such as alkyl and alkylaryl radicals. The aliphatic radicals may contain heteroatoms, such as oxygen, nitrogen, sulphur, and halogens. The aliphatic radicals may be chosen, for example, from alkyl, alkoxy, polyoxyalkylene (C₂-C₆), alkylamide, alkyl(C₁₂-C₂₂) amidoalkyl(C₂-C₆), alkyl(C₁₂-C₂₂)acetate, and hydroxyalkyl radicals containing 1 to 30 carbon atoms.

X is an anion chosen from halides, phosphates, acetates, lactates, C₂-C₆ alkyl sulphates, alkylsulphonates and alkylarylsulphonates;
quaternary ammonium salts of imidazoline, such as, for example, those of the formula (II) below: 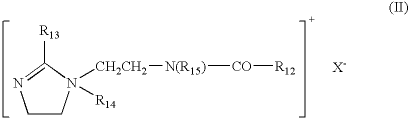
   in which

R₁₂ is chosen from alkenyl and alkyl radicals containing 8 to 30 carbon atoms, derived for example from tallow fatty acids;

R₁₃ is chosen from hydrogen, C₁-C₄ alkyl radicals, alkenyl radicals containing 8 to 30 carbon atoms, and alkyl radicals containing 8 to 30 carbon atoms;

R₁₄ is chosen from C₁-C₄ alkyl radicals,

R₁₅ is chosen from hydrogen and C₁-C₄ alkyl radicals; and

X⁻ is an anion chosen from halides, phosphates, acetates, lactates, alkyl sulphates and alkylsulphonates, and alkylarylsulphonates.

In at least one embodiment, R₁₂ and R₁₃ are a mixture of alkenyl or alkyl radicals containing 12 to 21 carbon atoms, derived, for example, from tallow fatty acids; R₁₄ is a methyl radical; and R₁₅ is a hydrogen atom. A product of this kind is sold, for example, under the name REWOQUAT W 75 by Rewo;

quaternary diammonium salts of formula (III): 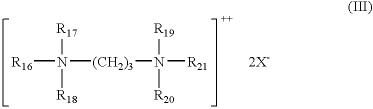
in which

R₁₆ is an aliphatic radical containing 16 to 30 carbon atoms;

R₁₇, R₁₈, R₁₉, R₂₀, and R₂₁, which may be identical or different, are chosen from hydrogen and alkyl radicals containing 1 to 4 carbon atoms; and

X is an anion chosen from halides, acetates, phosphates, nitrates, and methyl sulphates. Quaternary diammonium salts of this kind include propane tallow diammonium dichloride;

quaternary ammonium salts containing at least one ester function, such as those of formula (IV): 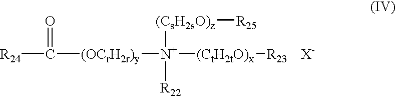
in which:

R₂₂ is chosen from C₁-C₆ alkyl radicals, C₁-C₆ hydroxyalkyl, and C₁-C₆ dihydroxyalkyl radicals;

R₂₃ is chosen from:
the radicals 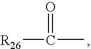
linear or branched, saturated or unsaturated C₁-C₂₂ hydrocarbon radicals R₂₇, and hydrogen;

R₂₅ is chosen from:
the radicals 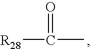
linear or branched, saturated or unsaturated C₁-C₆ hydrocarbon radicals R₂₉, and hydrogen;

R₂₄, R₂₆, and R₂₈, which may be identical or different, are chosen from linear or branched, saturated or unsaturated C₇-C₂₁ hydrocarbon radicals;

r, s, and t, which are identical or different, are chosen from integers ranging from 2 to 6;

y is chosen from integers ranging from 1 to 10;

x and z, which are identical or different, are chosen from integers ranging from 0 to 10;

X⁻ is chosen from simple or complex, organic or inorganic anions;

with the provisos that the sum x+y+z has a value ranging from 1 to 15, that when x is 0 then R₂₃ denotes R₂₇, and that when z is 0 then R₂₅ denotes R₂₉.

The alkyl radical R₂₂ may be linear or branched, and in at least one embodiment is linear. In certain embodiments, R₂₂ is chosen from methyl, ethyl, hydroxyethyl, and dihydroxypropyl radicals, for example methyl and ethyl radicals.

In certain embodiments, the sum x+y+z has a value ranging from 1 to 10.

When R₂₃ is a hydrocarbon radical R₂₇, it may be long and may have 12 to 22 carbon atoms or may be short and may have 1 to 3 carbon atoms.

When R₂₅ is a hydrocarbon radical R₂₉ it may have 1 to 3 carbon atoms.

In certain embodiments, R₂₄, R₂₆ and R₂₈, which may be identical or different, are chosen from linear or branched, saturated or unsaturated C₁₁-C₂₁ hydrocarbon radicals, such as linear or branched, saturated or unsaturated C₁₁-C₂₁ alkyl and alkenyl radicals.

In certain embodiments, x and z, which may be identical or different, are chosen from 0 and 1.

In certain embodiments, y is 1.

In yet further embodiments, r, s and t, which may be identical or different, are chosen from 2 and 3.

The anion X⁻ may be chosen from halides (such as chloride, bromide, or iodide) and alkyl sulphates, such as methyl sulphate. The anion may also be chosen from methanesulphonate, phosphate, nitrate, tosylate, anions derived from an organic acid, such as acetate or lactate, and any other ions which are compatible with the ester-functional ammonium.

The anion X⁻ may, for example, be chosen from chloride and methyl sulphate.

In the composition disclosed herein, mention may be made of the ammonium salts of formula (IV) in which:

R₂₂ is chosen from methyl and ethyl radicals,

x and y are 1;

z is chosen from 0 and 1;

r, s, and t are 2;

R₂₃ is chosen from:

the radicals 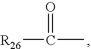
methyl, ethyl, and C₁₄-C₂₂ hydrocarbon radicals, and hydrogen;

R₂₅ is chosen from:

the radicals 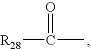
and hydrogen;

R₂₄, R₂₆, and R₂₈, which are identical or different, are chosen from linear or branched, saturated or unsaturated C₁₃-C₁₇ hydrocarbon radicals, such as linear or branched, saturated or unsaturated C₁₃-C₁₇ alkyl and alkenyl radicals.

In certain embodiments the hydrocarbon radicals are linear.

Mention may be made, for example, of the compounds of formula (IV) such as the salts (for example chloride sulphate and methyl sulphate) of diacyloxyethyldimethylammonium, diacyloxyethylhydroxyethylmethylammonium, monoacyloxyethyldihydroxyethylmethylammonium, triacyloxyethylmethylammonium, monoacyloxyethylhydroxyethyldimethylammonium, and mixtures thereof. The acyl radicals may have 14 to 18 carbon atoms and may originate from a vegetable oil, such as palm oil or sunflower oil. When the compound contains two or more acyl radicals, the acyl radicals may be identical or different.

These products may be obtained, for example, by direct esterification of triethanolamine, triisopropanolamine, alkyldiethanolamine, or alkyldiisopropanolamine, which are optionally oxyalkylenated, with fatty acids or with fatty acid mixtures of vegetable or animal origin, or by transesterification of their methyl esters. This esterification may be followed by quaternization by means of an alkylating agent such as an alkyl (for example methyl or ethyl)halide, a dialkyl (for example methyl or ethyl)sulphate, methyl methanesulphonate, methyl para-toluenesulphonate, glycol chlorohydrin, or glycerol chlorohydrin.

Compounds of this kind are sold, for example, under the names Dehyquart® by Henkel, Stepanquat® by Stepan, NOXAMIUM by Ceca, and REWOQUAT WE 18 by Rewo-Witco.

The composition disclosed herein may contain a mixture of monoester, diester, and triester salts of quaternary ammonium, with a majority by weight of diester salts.

As an example of a mixture of ammonium salts, mention may be made of the mixture containing 15% to 30% by weight of acyloxyethyldihydroxyethylmethylammonium methyl sulphate, 45% to 60% of diacyloxyethyl-hydroxyethylmethylammonium methyl sulphate, and 15% to 30% of triacyloxyethylmethylammonium methyl sulphate, the acyl radicals having 14 to 18 carbon atoms and originating from palm oil, which has optionally been partially hydrogenated.

Mention may also be made of the ammonium salts containing at least one ester function that are described, for example, in U.S. Pat. Nos. 4,874,554 and 4,137,180.

Among the quaternary ammonium salts of formula (I), mention may be made of tetraalkylammonium chlorides such as, for example, dialkyldimethylammonium and alkyltrimethylammonium chlorides in which the alkyl radical contains 12 to 22 carbon atoms, such as behenyltrimethylammonium, distearyldimethylammonium, cetyltrimethylammonium, and benzyldimethylstearylammonium chlorides. Mention may also be made of palmitylamidopropyltrimethylammonium chloride and stearamidopropyl-dimethyl(myristyl acetato)ammonium chloride, which is sold under the name Ceraphyl® 70 by Van Dyk.

The cationic surfactants which may be used in the composition disclosed herein may be chosen from quaternary ammonium salts, such as cetyltrimethylammonium chloride, behenyltrimethylammonium chloride, and palmitylamidopropyltrimethylammonium chloride.

The ionic surfactants which can be used in the cosmetic composition disclosed herein may also be chosen from anionic surfactants.

Anionic surfactants which can be used in the cosmetic composition disclosed herein include, for example, salts, such as the alkali metal salts such as sodium salts; the ammonium salts; the amine salts; the amino alcohol salt; and the alkaline earth metal salts, for example magnesium salts, of the following types: alkyl sulphates, alkyl ether sulphates, alkylamido ether sulphates, alkylaryl polyether sulphates, monoglyceride sulphates, alkylsulphonates, alkylamidesulphonates, alkylarylsulphonates, α-olefinesulphonates, paraffinsulphonates, alkylsulphosuccinates, alkyl ether sulphosuccinates, alkylamidesulphosuccinates, alkylsulphoacetates, acylsarcosinates, and acylglutamates, wherein the alkyl and acyl groups of all of these compounds contain 6 to 24 carbon atoms and the aryl group optionally being chosen from phenyl and benzyl groups.

It is also possible to use the C₆₋₂₄ alkyl monoesters of polyglycoside-dicarboxylic acids, such as alkyl glucoside-citrates, alkyl polyglycoside-tartrates, alkyl polyglycoside-sulphosuccinates, alkylsulphosuccinamates, acylisethionates, and N-acyltaurates, the alkyl or acyl group of all of these compounds containing 12 to 20 carbon atoms.

Another group of anionic surfactants which can be used in the compositions disclosed herein is that of acyllactylates whose acyl group contains 8 to 20 carbon atoms.

In addition, mention may also be made of alkyl-D-galactosideuronic acids and their salts, and also of polyoxyalkylenated alkyl(C₆₋₂₄)ethercarboxylic acids, polyoxyalkylenated alkyl(C₆₋₂₄)aryl(C₆₋₂₄)ethercarboxylic acids, polyoxyalkylenated alkyl(C₆-C₂₄)amidoethercarboxylic acids and their salts, for example those containing from 2 to 50 ethylene oxide units, and mixtures thereof.

In certain embodiments, anionic surfactants which can be used in the compositions disclosed herein are chosen from at least one of alkyl sulphates, alkyl ether sulphates, and alkyl ether carboxylates, for example in the form of salts with at least one entity chosen from alkali metals, alkaline earth metals, ammonium, amines, and amino alcohols.

The ionic surfactant may be chosen from cationic surfactants.

The nonionic surfactants which can be used in the cosmetic composition disclosed herein are compounds which may be well known per se (see in particular in this respect “Handbook of Surfactants” by M. R. Porter, published by Blackie & Son (Glasgow and London), 1991, pp 116-178). They may be chosen from alcohols, alpha-diols, alkyl(C₁₋₂₀)phenols, and fatty acids which are polyethoxylated, polypropoxylated, or polyglycerolated, having a fatty chain containing, for example, 8 to 18 carbon atoms, it being possible for the number of ethylene oxide or propylene oxide groups to range from 2 to 50 and for the number of glycerol groups to range from 2 to 30.

Mention may also be made of condensates of ethylene oxide and of propylene oxide with fatty alcohols; polyethoxylated fatty amides having, for example, from 2 to 30 ethylene oxide units; polyglycerolated fatty amides containing on average from 1 to 5 glycerol groups, such as from 1.5 to 4 glycerol groups; ethoxylated fatty acid esters of sorbitan having from 2 to 30 ethylene oxide units; fatty acid esters of sucrose; fatty acid esters of polyethylene glycol; (C₆₋₂₄ alkyl)polyglycosides; N—(C₆₋₂₄ alkyl)glucamine derivatives; and amine oxides such as (C₁₀₋₁₄ alkyl)amine oxides and N—(C₁₀-C₁₄ acyl)aminopropylmorpholine oxides.

Among the nonionic surfactants mentioned above, mention may be made of polyethoxylated, polypropoxylated, and polyglycerolated alcohols.

The at least one surfactant may be present in an amount ranging from 0.01% to 20% by weight, such as from 0.05% to 10% by weight or from 0.1% to 5% by weight, relative to the total weight of the composition.

The at least one fixative polymer as disclosed herein may be any polymer which allows a shape to be imparted or a given shape or hairstyle to be retained.

The ethylenically unsaturated carboxylic acid may be chosen from acrylic acid, methacrylic acid, itaconic acid, and crotonic acid.

The ethylenically unsaturated carboxylic acid may, for example, be chosen from acrylic acid and methacrylic acid. In certain embodiments, the ethylenically unsaturated carboxylic acid is acrylic acid.

The vinyl lactam may be vinylpyrrolidone.

The alkyl acrylate or alkyl methacrylate containing at least 6 carbon atoms may be chosen from acrylic esters and methacrylic esters having an alkyl radical containing 8 to 18 carbon atoms.

The alkyl radicals may be chosen from 2-ethylhexyl, octyl, lauryl, and stearyl radicals.

These polymers may be optionally crosslinked.

The at least one anionic fixative polymer which can be used in the composition disclosed herein may be prepared, for example, according to the process described in U.S. Pat. No. 5,015,708.

The at least one anionic fixative polymer which can be used in the composition disclosed herein may comprise as monomers at least one of vinylpyrrolidone, acrylic acid, and lauryl methacrylate.

In one embodiment, the polymer disclosed herein is the polymer sold under the name Acrylidone® LM by ISP, which is a terpolymer of vinylpyrrolidone (68%)/acrylic acid (23%) and lauryl methacrylate (9%), the percentages by weight of each of the monomers being calculated relative to the total weight of the at least one anionic fixative polymer.

The at least one anionic fixative polymer based on vinyl lactam, ethylenically unsaturated acid, and alkyl acrylate or alkyl methacrylate may be present in the composition in an amount ranging from 0.05% to 30% by weight, such as from 0.1% to 20% by weight or from 0.5% to 10% by weight, relative to the total weight of the composition.

The weight ratio between the at least one anionic fixative polymer disclosed herein and the at least one surfactant chosen from ionic and nonionic surfactants disclosed herein may be greater than 1.

One embodiment of the cosmetic composition disclosed herein comprises a fixative polymer which is based on vinylpyrrolidone, acrylic acid, and lauryl methacrylate and is present in an amount of less than 3.5% by weight and a cationic surfactant which is present in an amount of less than 1% by weight, the percentages being calculated by weight relative to the total weight of the cosmetic composition.

This composition may be packaged in a pump dispenser bottle or in an aerosol device. The compositions disclosed herein which are packed in these devices lead to high-quality sprays.

The cosmetic compositions disclosed herein may exhibit a viscosity ranging from 2 to 1,000 poise (200 to 100,000 cps) at 25° C., such as from 5 to 500 poise (500 to 50,000 cps) at 25° C. or from 8 to 300 poise (800 to 30,000 cps) at 25° C. at a shear rate of 1 s⁻¹, measured by means of the RS600 rheometer from Thermoelectron.

In one embodiment, the compositions disclosed herein do not comprise any additional thickening polymers.

The cosmetic composition disclosed herein may further comprise at least one cosmetic adjuvant chosen from fixative polymers other than those disclosed herein; dispersed, microdispersed, and nanodispersed silicones in soluble form; non-polymeric thickeners; thickening polymers and gelling agents which are not sulphonated; cationic, anionic, amphoteric, and nonionic surfactants; ester-type conditioning agents; antifoams; moisturizers; emollients; plasticizers; water-soluble and fat-soluble silicone or non-silicone sunscreens; permanent or temporary dyes; fragrances; peptizing agents; preservatives; ceramides and pseudoceramides; vitamins and provitamins, including panthenol, proteins, sequestrants, solubilizers, alkalifiers, anticorrosives, fatty substances such as vegetable, animal, mineral, and synthetic oils, reducing agents and antioxidants; and oxidizing agents.

The person skilled in the art will ensure that any adjuvants and their amount are selected in such a way as to cause no detriment to the properties of the compositions disclosed herein.

The at least one cosmetic adjuvant may be present in an amount ranging from 0.001% to 50% by weight, relative to the total weight of the composition.

A cosmetically acceptable medium is a medium which is compatible with keratin material, such as the hair.

The cosmetically acceptable medium may be chosen from aqueous, alcoholic, and aqueous-alcoholic mediums. Thus the medium may be composed solely of water, or solely of alcohol, or it may be composed of a mixture of water and at least one cosmetically acceptable solvent, such as C₁-C₄ lower alcohols, polyols, and polyol monoethers.

The compositions disclosed herein may take the form of a gel and may be used in rinse-off or leave-in applications.

These compositions may be packaged, for example, in a vaporizer, in a pump dispenser bottle, or in an aerosol device customary in cosmetology.

The propellants used in the aerosol systems disclosed herein may be chosen from at least one of air, nitrogen, carbon dioxide, dimethyl ether, C₃ to C₅ alkanes, and 1,1-difluoroethane.

Also disclosed herein is an aerosol device comprising the composition disclosed herein and a means of distributing this composition.

Another embodiment disclosed herein is a cosmetic treatment method for the hair, for example a hairstyling method, comprising applying an effective amount of the composition disclosed herein to wet or dry hair, and rinsing the composition off or leaving it in after an optional exposure time or an optional drying. One mode of application is a leave-in mode.

Further disclosed herein is the use of a cosmetic composition for shaping and/or retaining the hairstyle.

The examples which follow are given by way of illustration and not of limitation of the present invention.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible.

### EXAMPLES

Styling gels (A), (B) and (C) according to the invention were prepared from the following components:

Composition (A)

| | |
|---|---|
| Vinylpyrrolidone/acrylic acid/lauryl methacrylate | 2% |
| terpolymer provided by ISP under the name | |
| Acrylidone ® LM | |
| Steareth-20 | 0.01% |
| (nonionic surfactant) | |
| Cremophor RH-40 | 1% |
| (nonionic surfactant) | |
| Preservatives | qs |
| Water qs | 100% |

The composition gave a styling gel (A) having a viscosity of 50 poise (5,000 cps) at 25° C. as measured on the RS600 rheometer from Thermoelectron at a shear rate of 1 s⁻¹.

Composition (B)

| | |
|---|---|
| Vinylpyrrolidone/acrylic acid/lauryl methacrylate | 2% |
| terpolymer provided by ISP under the name | |
| Acrylidone ® LM | |
| Behenyltrimethylammonium chloride | 0.5% |
| (cationic surfactant) | |
| Preservatives | qs |
| Water qs | 100% |

The composition gave a styling gel (B) having a viscosity of 16 poise (1,600 cps) at 25° C. as measured on the RS600 rheometer from Thermoelectron at a shear rate of 1 s⁻¹.

Composition (C)

| | |
|---|---|
| Vinylpyrrolidone/acrylic acid/lauryl methacrylate | 4% |
| terpolymer provided by ISP under the name | |
| Acrylidone ® LM | |
| Lauryl ether sulphate containing 2.2 mol of EO | 2% |
| (anionic surfactant) | |
| Preservatives | qs |
| Water qs | 100% |

The composition gave a styling gel (C) having a viscosity of 150 poise (15,000 cps) at 25° C. as measured on the RS600 rheometer from Thermoelectron at a shear rate of 1 s⁻¹.

The percentages of each of the compounds in the styling gels (A), (B), and (C) were calculated by weight relative to the total weight of the composition.

The resulting styling gels (A), (B), and (C) were applied to the hair.

The performances of these styling gels were evaluated by professionals.

It is found that styling gels (A), B, and (C) produced effective fixation while at the same time allowing the formation of unattractive residues on the body of hair to be minimized.

It is further observed that, when styling gel (B) was packaged in a pump dispenser bottle, a highly satisfactory spray was obtained.

What is claimed is:

1. A cosmetic composition for treating keratin fibers, comprising, in a cosmetically acceptable medium,
at least one anionic fixative polymer comprising as monomers at least a vinyl lactam, an ethylenically unsaturated carboxylic acid, and an acrylate chosen from alkyl acrylates and alkyl methacrylates, wherein alkyl moiety contains at least 6 carbon atoms; and
at least one surfactant chosen from ionic and nonionic surfactants;
wherein the cosmetic composition exhibits a viscosity greater than 2 poise (200 cps) at 25° C. at a shear rate of 1 s⁻¹.

2. The cosmetic composition according to claim 1, wherein the at least one ionic surfactant is chosen from cationic surfactants.

3. The cosmetic composition according to claim 2, wherein the at least one cationic surfactant is chosen from the salts of optionally polyoxyalkylenated primary, secondary, or tertiary fatty amines, quaternary ammonium salts, and mixtures thereof.

4. The cosmetic composition according to claim 3, wherein the quaternary ammonium salts are chosen from:
those having formula (I) below: 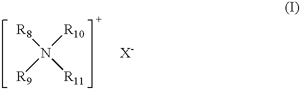
in which the radicals R₈ to R₁₁, which may be identical or different, are chosen from linear or branched aliphatic radicals comprising 1 to 30 carbon atoms and aromatic radicals; and X is an anion chosen from halides, phosphates, acetates, lactates, C₂-C₆ alkyl sulphates, alkylsulphonates and alkylarylsulphonates;
quaternary ammonium salts of imidazoline;
quaternary diammonium salts of formula (II): 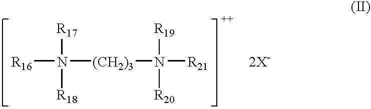
in which R₁₆ is an aliphatic radical comprising 16 to 30 carbon atoms, R₁₇, R₁₈, R₁₉, R₂₀ and R₂₁, which are identical or different, are chosen from hydrogen and alkyl radicals comprising 1 to 4 carbon atoms, and X is an anion chosen from halides, acetates, phosphates, nitrates, and methyl sulphates; and
quaternary ammonium salts containing at least one ester function.

5. The cosmetic composition according to claim 4, wherein in formula (I), R₈ to R₁₁ are aromatic radicals chosen from aryl or alkylaryl radicals.

6. The cosmetic composition according to claim 1, wherein the at least one ionic surfactant is chosen from anionic surfactants chosen from alkyl sulphates, alkyl ether sulphates, and alkyl ether carboxylates.

7. The cosmetic composition according to claim 6, wherein the at least one anionic surfactant is in the form of a salt with at least one of alkali metals, alkaline earth metals, ammonium, amines, and amino alcohols.

8. The cosmetic composition according to claim 1, wherein the at least one nonionic surfactant is chosen from alcohols; alpha-diols; alkylphenols; fatty acids which are polyethoxylated, polypropoxylated, or polyglycerolated, having a fatty chain comprising 8 to 18 carbon atoms, wherein the number of ethylene oxide and propylene oxide groups optionally ranges from 2 to 50 and the number of glycerol groups optionally ranges from 2 to 30; fatty acid esters of sucrose; fatty acid esters of polyethylene glycol; (C₆₋₂₄ alkyl)polyglucosides; N—(C₆₋₂₄ alkyl)glucamine derivatives; C₁₀-C₁₄ alkylamine oxides; and N—(C₁₀₋₁₄ acyl)aminopropylmorpholine oxides.

9. The cosmetic composition according to claim 1, wherein the unsaturated carboxylic acid is chosen from acrylic acid, methacrylic acid, itaconic acid, and crotonic acid.

10. The cosmetic composition according to claim 9, wherein the unsaturated carboxylic acid is acrylic acid.

11. The cosmetic composition according to claim 1, wherein the vinyl lactam is vinylpyrrolidone.

12. The cosmetic composition according to claim 1, wherein the acrylate chosen from alkyl acrylates and alkyl methacrylates comprises an alkyl radical comprising 8 to 18 carbon atoms.

13. The cosmetic composition according to claim 12, wherein the alkyl radical of said acrylates is chosen from 2-ethylhexyl, octyl, lauryl, and stearyl radicals.

14. The cosmetic composition according to claim 1, wherein the at least one anionic fixative polymer comprises as monomers vinylpyrrolidone, acrylic acid, and lauryl methacrylate.

15. The cosmetic composition according to claim 1, wherein the at least one anionic fixative polymer is present in an amount ranging from 0.05% to 30% by weight, relative to the total weight of the composition.

16. The cosmetic composition according to claim 15, wherein the at least one anionic fixative polymer is present in an amount ranging from 0.1% to 20% by weight, relative to the total weight of the composition.

17. The cosmetic composition according to claim 16, wherein the at least one anionic fixative polymer is present in an amount ranging from 0.5% to 10% by weight, relative to the total weight of the composition.

18. The cosmetic composition according to claim 1, wherein the at least one surfactant is present in an amount ranging from 0.01% to 20% by weight, relative to the total weight of the composition.

19. The cosmetic composition according to claim 18, wherein the at least one surfactant is present in an amount ranging from 0.05% to 10% by weight, relative to the total weight of the composition.

20. The cosmetic composition according to claim 19, wherein the at least one surfactant is present in an amount ranging from 0.1% to 5% by weight, relative to the total weight of the composition.

21. The cosmetic composition according to claim 1, wherein the composition exhibits a viscosity ranging from 2 to 1,000 poise (200 to 100,000 cps) at 25° C., at a shear rate of 1 s⁻¹.

22. The cosmetic composition according to claim 21, wherein the composition exhibits a viscosity ranging from 5 to 500 poise (500 to 50,000 cps) at 25° C., at a shear rate of 1 s⁻¹.

23. The cosmetic composition according to claim 22, wherein the composition exhibits a viscosity ranging from 8 to 300 poise (800 to 30,000 cps) at 25° C., at a shear rate of 1 s⁻¹.

24. The cosmetic composition according to claim 1, wherein the at least one anionic fixative polymer and the at least one surfactant chosen from ionic and nonionic surfactants are present in a weight ratio greater than 1.

25. The cosmetic composition according to claim 1, further comprising at least one cosmetic adjuvant chosen from fixative polymers; dispersed, microdispersed, or nanodispersed silicones in soluble form; non-polymeric thickeners; thickening polymers and gelling agents which are not sulphonated; cationic, anionic, amphoteric, and nonionic surfactants; ester-type conditioning agents; antifoams; moisturizers; emollients; plasticizers; water-soluble and fat-soluble silicone and non-silicone sunscreens; permanent dyes; temporary dyes; fragrances; peptizing agents; preservatives; ceramides; pseudoceramides; vitamins and provitamins; proteins; sequestrants; solubilizers; alkalifiers; anticorrosives; fatty substances; reducing agents; antioxidants; and oxidizing agents.

26. The cosmetic composition according to claim 25, wherein the at least one cosmetic adjuvant is present for each in an amount ranging from 0.001% to 50% by weight, relative to the total weight of the composition.

27. The cosmetic composition according to claim 1, wherein the cosmetically acceptable medium is chosen from aqueous, alcoholic, and aqueous-alcoholic mediums.

28. The cosmetic composition according to claim 27, wherein the aqueous-alcoholic medium comprises at least one of C₁-C₄ lower alcohols, polyols, and polyol monoethers.

29. The cosmetic composition according to claim 28, wherein the C₁-C₄ lower alcohol is ethanol.

30. The cosmetic composition according to claim 1, wherein the composition does not comprise any additional thickening polymers.

31. The cosmetic composition according to claim 1, wherein the composition is packaged in a device chosen from vaporizers, pump dispenser bottles, and aerosol devices.

32. The cosmetic composition according to claim 31, wherein the composition is packaged in an aerosol device.

33. The cosmetic composition according to claim 32, wherein the composition comprises a propellant chosen from at least one of air, nitrogen, carbon dioxide, dimethyl ether, C₃ to C₅ alkanes, and 1,1-difluoroethane.

34. The cosmetic composition according to claim 1, wherein the at least one anionic fixative polymer comprises as monomers vinylpyrrolidone, acrylic acid, and lauryl methacrylate, and wherein the at least one anionic fixative polymer is present in an amount of less than 3.5% by weight, and the at least one surfactant is at least one cationic surfactant present in an amount of less than 10% by weight, relative to the total weight of the composition, and the composition is packaged in device chosen from pump dispenser bottles and aerosol devices.

35. An aerosol device formed by a container comprising,
a cosmetic composition for treating keratin fibers comprising, in a cosmetically acceptable medium,
at least one anionic fixative polymer comprising as monomers at least a vinyl lactam, an ethylenically unsaturated carboxylic acid, and an acrylate chosen from alkyl acrylates and methacrylates, wherein alkyl moiety contains at least 6 carbon atoms; and
at least one surfactant chosen from ionic and nonionic surfactants;
wherein the cosmetic composition exhibits a viscosity greater than 2 poise (200 cps) at 25° C. at a shear rate of 1 s⁻¹, and
a means of distributing the cosmetic composition.

36. A method for shaping and/or retaining a hairstyle, comprising
applying a cosmetic composition for treating keratin fibers comprising, in a cosmetically acceptable medium,
at least one anionic fixative polymer comprising as monomers at least a vinyl lactam, an ethylenically unsaturated carboxylic acid, and an acrylate chosen from alkyl acrylates and methacrylates, wherein alkyl moiety contains at least 6 carbon atoms; and
at least one surfactant chosen from ionic and nonionic surfactants;
wherein the cosmetic composition exhibits a viscosity greater than 2 poise (200 cps) at 25° C. at a shear rate of 1 s⁻¹,
optionally rinsing said keratin fibers.

37. The method according to claim 36, wherein a rinsing operation does not follow the step of applying the cosmetic composition.
